SEQUENCE LISTING (45 sequences)

SEQ ID NO: 1 - protein, 1382 aa, Homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 2 - protein, 1382 aa, Homo sapiens
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTTTQGKLFFHYNPKLCLSEIHKMEEVSGTKGRQERNDIALKTNGDKASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 3 - protein, 190 aa, Homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQK

SEQ ID NO: 4 - protein, 120 aa, Homo sapiens
VCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPK

SEQ ID NO: 5 - protein, 160 aa, Homo sapiens
KVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCE

SEQ ID NO: 6 - protein, 125 aa, Homo sapiens
ENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDAT

SEQ ID NO: 7 - protein, 929 aa, Homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 8 - protein, 917 aa, Homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 9 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 10 - protein, 30 aa, Homo sapiens
FVNQHLCGSHLVEALYLVCGERGFFYTPKT

SEQ ID NO: 11 - protein, 21 aa, Homo sapiens
GIVEQCCTSICSLYQLENYCN

SEQ ID NO: 12 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 13 - protein, 63 aa, Homo sapiens
FVNQHLCGSHLVEALYLVCGERGFFYTPKTGYGSSSRRAPQTGIVEQCCTSICSLYQLENYCN

SEQ ID NO: 14 - protein, 58 aa, Homo sapiens
FVNQHLCGSHLVEALYLVCGERGFFYTPKTGGGPGKRGIVEQCCTSICSLYQLENYCN

SEQ ID NO: 15 - protein, 323 aa, Artificial
  proinsulin fused to L1 domain
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKGGGGSGGGGSGGGGSGGGGSFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 16 - protein, 300 aa, Artificial
  L1 domain fused to single insulin chain 1
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKGGGGSGGGGSGGGGSGGGGSFVNQHLCGSHLVEALYLVCGERGFFYTPKTGYGSSSRRAPQTGIVEQCCTSICSLYQLENYCN

SEQ ID NO: 17 - protein, 295 aa, Artificial
  L1 domain fused to single insulin chain 2
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKGGGGSGGGGSGGGGSGGGGSFVNQHLCGSHLVEALYLVCGERGFFYTPKTGGGPGKRGIVEQCCTSICSLYQLENYCN

SEQ ID NO: 18 - protein, 293 aa, Artificial
  L2 domain fused to proinsulin
MGTGGRRGAAAAPLLVAVAALLLGAAGKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCEGGGGSGGGGSGGGGSGGGGSFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 19 - protein, 270 aa, Artificial
  L2 domain fused to single chain insulin 1
MGTGGRRGAAAAPLLVAVAALLLGAAGKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCEGGGGSGGGGSGGGGSGGGGSFVNQHLCGSHLVEALYLVCGERGFFYTPKTGYGSSSRRAPQTGIVEQCCTSICSLYQLENYCN

SEQ ID NO: 20 - protein, 265 aa, Artificial
  L2 domain fused to insulin single chain 2
MGTGGRRGAAAAPLLVAVAALLLGAAGKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCEGGGGSGGGGSGGGGSGGGGSFVNQHLCGSHLVEALYLVCGERGFFYTPKTGGGPGKRGIVEQCCTSICSLYQLENYCN

SEQ ID NO: 21 - protein, 258 aa, Artificial
  FnIII domain fused to proinsulin
MGTGGRRGAAAAPLLVAVAALLLGAAGENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATGGGGSGGGGSGGGGSGGGGSFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 22 - protein, 235 aa, Artificial
  FnIII domain fused to single chain insulin 1
MGTGGRRGAAAAPLLVAVAALLLGAAGENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATGGGGSGGGGSGGGGSGGGGSFVNQHLCGSHLVEALYLVCGERGFFYTPKTGYGSSSRRAPQTGIVEQCCTSICSLYQLENYCN

SEQ ID NO: 23 - protein, 230 aa, Artificial
  FnIII domain fused to insulin single chain 2
MGTGGRRGAAAAPLLVAVAALLLGAAGENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATGGGGSGGGGSGGGGSGGGGSFVNQHLCGSHLVEALYLVCGERGFFYTPKTGGGPGKRGIVEQCCTSICSLYQLENYCN

SEQ ID NO: 24 - protein, 1082 aa, Artificial [sequence omitted]

SEQ ID NO: 25 - protein, 1059 aa, Artificial
  extracellular domain of isoform B fused to single chain insulin 1
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKGGGGSGGGGSGGGGSGGGGSGGGGSGGGGSGGGGSGGGGSFVNQHLCGSHLVEALYLVCGERGFFYTPKTGYGSSSRRAPQTGIVEQCCTSICSLYQLENYCN

SEQ ID NO: 26 - protein, 1054 aa, Artificial
  extracellular domain of isoform b fused to insulin single chain 2
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKGGGGSGGGGSGGGGSGGGGSGGGGSGGGGSGGGGSGGGGSFVNQHLCGSHLVEALYLVCGERGFFYTPKTGGGPGKRGIVEQCCTSICSLYQLENYCN

SEQ ID NO: 27 - protein, 1070 aa, Artificial
  extracellular domain of isoform A linked to proinsulin
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKGGGGSGGGGSGGGGSGGGGSGGGGSGGGGSGGGGSGGGGSFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 28 - protein, 1047 aa, Artificial
  single chain insulin fused to L1 domain
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKGGGGSGGGGSGGGGSGGGGSGGGGSGGGGSGGGGSGGGGSFVNQHLCGSHLVEALYLVCGERGFFYTPKTGYGSSSRRAPQTGIVEQCCTSICSLYQLENYCN

SEQ ID NO: 29 - protein, 1047 aa, Artificial; identical to SEQ ID NO: 28
  extracellular domain of isoform A linked to single chain insulin 1

SEQ ID NO: 30 - protein, 1042 aa, Artificial
  extracellular domain of isoform A linked to single chain insulin 2
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKGGGGSGGGGSGGGGSGGGGSGGGGSGGGGSGGGGSGGGGSFVNQHLCGSHLVEALYLVCGERGFFYTPKTGGGPGKRGIVEQCCTSICSLYQLENYCN

SEQ ID NO: 31 - protein, 320 aa, Artificial
  proinsulin linked to L1 domain
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQK

SEQ ID NO: 32 - protein, 297 aa, Artificial
  single chain insulin 1 linked to L1 domain
MALWMRLLPLLALLALWGPDPAAAFVNQHLCGSHLVEALYLVCGERGFFYTPKTGYGSSSRRAPQTGIVEQCCTSICSLYQLENYCNGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQK

SEQ ID NO: 33 - protein, 292 aa, Artificial
  single chain insulin 2 linked to L1 domain
MALWMRLLPLLALLALWGPDPAAAFVNQHLCGSHLVEALYLVCGERGFFYTPKTGGGPGKRGIVEQCCTSICSLYQLENYCNGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQK

SEQ ID NO: 34 - protein, 290 aa, Artificial
  proinsulin linked to L2 domain
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNGGGGSGGGGSGGGGSGGGGSKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCE

SEQ ID NO: 35 - protein, 267 aa, Artificial
  single chain insulin linked to L2 domain
MALWMRLLPLLALLALWGPDPAAAFVNQHLCGSHLVEALYLVCGERGFFYTPKTGYGSSSRRAPQTGIVEQCCTSICSLYQLENYCNGGGGSGGGGSGGGGSGGGGSKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCE

SEQ ID NO: 36 - protein, 262 aa, Artificial
  single chain insulin 2 linked to L2 domain
MALWMRLLPLLALLALWGPDPAAAFVNQHLCGSHLVEALYLVCGERGFFYTPKTGGGPGKRGIVEQCCTSICSLYQLENYCNGGGGSGGGGSGGGGSGGGGSKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCE

SEQ ID NO: 37 - protein, 255 aa, Artificial
  proinsulin fused to FnIII domain
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNGGGGSGGGGSGGGGSGGGGSENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDAT

SEQ ID NO: 38 - protein, 232 aa, Artificial
  single chain insulin 1 fused to FnIII domain
MALWMRLLPLLALLALWGPDPAAAFVNQHLCGSHLVEALYLVCGERGFFYTPKTGYGSSSRRAPQTGIVEQCCTSICSLYQLENYCNGGGGSGGGGSGGGGSGGGGSENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDAT

SEQ ID NO: 39 - protein, 227 aa, Artificial
  single chain insulin 2 fused to FnIII domain
MALWMRLLPLLALLALWGPDPAAAFVNQHLCGSHLVEALYLVCGERGFFYTPKTGGGPGKRGIVEQCCTSICSLYQLENYCNGGGGSGGGGSGGGGSGGGGSENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDAT

SEQ ID NO: 40 - protein, 1079 aa, Artificial [sequence omitted]

SEQ ID NO: 41 - protein, 1056 aa, Artificial
  single chain insulin 1 fused to extracellular domain isoform B
MALWMRLLPLLALLALWGPDPAAAFVNQHLCGSHLVEALYLVCGERGFFYTPKTGYGSSSRRAPQTGIVEQCCTSICSLYQLENYCNGGGGSGGGGSGGGGSGGGGSGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 42 - protein, 1051 aa, Artificial
  single chain insulin 2 linked to extracellular domain isoform B
MALWMRLLPLLALLALWGPDPAAAFVNQHLCGSHLVEALYLVCGERGFFYTPKTGGGPGKRGIVEQCCTSICSLYQLENYCNGGGGSGGGGSGGGGSGGGGSGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 43 - protein, 1067 aa, Artificial
  proinsulin fused to extracellular domain isoform A
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNGGGGSGGGGSGGGGSGGGGSGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 44 - protein, 1044 aa, Artificial
  single chain insulin 1 fused to extracellular domain isoform A
MALWMRLLPLLALLALWGPDPAAAFVNQHLCGSHLVEALYLVCGERGFFYTPKTGYGSSSRRAPQTGIVEQCCTSICSLYQLENYCNGGGGSGGGGSGGGGSGGGGSGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

SEQ ID NO: 45 - protein, 1039 aa, Artificial
  single chain insulin 2 linked to extracellular domain isoform A
MALWMRLLPLLALLALWGPDPAAAFVNQHLCGSHLVEALYLVCGERGFFYTPKTGGGPGKRGIVEQCCTSICSLYQLENYCNGGGGSGGGGSGGGGSGGGGSGGGGSGGGGSGGGGSGGGGSHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAK

(19) United States
(12) Patent Application Publication
(10) Pub. No.: US 2011/0230401 A1
(43) Pub. Date: Sep. 22, 2011

(54) INSULIN FUSION POLYPEPTIDES

(76) Inventors: Peter Artymiuk, Sheffield (GB); Richard Ross, Sheffield (GB)

(21) Appl. No.: 13/002,039

(22) PCT Filed: Jul. 2, 2009
(86) PCT No.: PCT/GB09/01668

§ 371 Date: Feb. 28, 2011

(30) Foreign Application Priority Data
Jul. 2, 2008 (GB) 0812019.8
Jul. 7, 2008 (US) 61078685

(51) Int. Cl. A61K 38/28; C07H 21/04; C07K 14/62; C12N 15/63; A61P 3/10; C12N 5/10; C12N 1/00

(52) U.S. Cl. 514/5.9; 536/23.4; 530/303; 530/350; 435/320.1; 435/325; 435/419; 435/243

(57) ABSTRACT

We disclose insulin fusion polypeptides and dimers; nucleic acid molecules encoding said polypeptides and methods of treatment that use said polypeptides/dimers.

The invention relates to insulin fusion polypeptides and dimers; nucleic acid molecules encoding said polypeptides and methods of treatment that use said polypeptides/dimers.

The interaction between proteins is fundamental to function and results in biological effects in cells such as regulation of energy metabolism, cell differentiation and cell proliferation. Proteins that interact with receptors to bring about a biochemical response are known as agonists and those that prevent, or hinder, a biochemical response are known as antagonists. Activation of the receptors by protein-specific binding promotes cell proliferation via activation of intracellular signalling cascades that result in the expression of, amongst other things, cell-cycle specific genes and the activation of quiescent cells to proliferate.

Insulin is an example of a protein that mediates activation of biochemical responses through receptors. Insulin functions to regulate glucose homeostasis. In conditions of hyperglycemia [abnormally high levels of serum glucose] the pancreatic β cells of the Islets of Langerhans synthesize proinsulin which is enzymatically cleaved at its amino and carboxy-termini to produce insulin, a 51 amino acid polypeptide. Insulin is secreted and acts on target cells [e.g. liver, muscle, adipose tissue] that express insulin receptors. The activation of insulin receptors leads to a signal transduction cascade that results in expression of glucose transporters which remove excess glucose receptors and convert the glucose into glycogen for storage. Once glucose levels return to normal insulin is degraded thus removing its biological effects. The insulin receptor is a tyrosine kinase and is a tetrameric transmembrane receptor comprising two α subunits and two β subunits. The α subunits are extracellular and bind insulin. The β subunits are transmembrane and include ATP and tyrosine kinase domains which become activated on insulin binding. The α and β subunits are linked to one another via disulphide bonds.

There are a number of pathological conditions that result in hyperglycaemia; the most well known being diabetes mellitus. Diabetes mellitus can be of type 1 or type 2. Type 1 diabetes is an autoimmune disease resulting in destruction of the pancreatic β cells which means the subject is unable to manufacture any insulin. Type 2 diabetes is a more complicated condition and can result from a number of associated ailments but commonly involves resistance to the metabolic actions of insulin. For example, type 2 diabetes is associated with age, obesity, a sedentary life style which results in insulin resistance. An associated condition is called Metabolic Syndrome which may predispose subjects to type 2 diabetes. The symptoms associated with this syndrome are high blood pressure, dyslipidemia, increased body fat deposition and cardiovascular disease. A further condition that results in insulin resistance is polycystic ovary syndrome which results in a failure to produce mature ova, androgen excess and hirsuitism. Hypoglycaemia [abnormally low levels of serum glucose] is also known and is typically the result of administration of an insulin overdose. However there are also diseases that result in excess insulin secretion resulting in a hypoglycaemic state. For example, insulinoma is a cancer of the pancreatic β cells resulting in over production of insulin.

Administration of insulin is an effective means to control conditions such as type 1 and type 2 diabetes. Historically insulin extracted from non-human sources have been used in the treatment of diabetes. Mammalian insulins are highly conserved and able to activate insulin receptors expressed by target cells. Recombinant human insulin is manufactured and is the preferred insulin for the treatment of hyperglycemia. A number of problems are associated with the use of insulin to control glucose metabolism. These include the mode of administration, dosage and type of insulin. A number of forms of insulin are known in the art which are differentiated from each other by the release and activity profile of the insulin or insulin variant. For example there are immediate acting [5-15 mins] medium release [3-4 hrs] forms; delayed acting [30 mins] moderate release [5-8 hrs] forms and delayed acting [4-6 hrs], sustained release [24-28 hrs] forms. These are insulins that modify the native insulin amino acid sequence to engineer an activity/release profile. A major side-effect of insulin therapy is hypoglycaemia and there is a need for a long-acting insulin analogue that provides sustained biological activity with low risk of hypoglycaemia.

We disclose native insulin in the form of an insulin: receptor fusion protein which has altered pharmakokinetic profile and activity. The insulin molecules are biologically active, form dimers and have improved serum stability. It will be apparent that the fusion technology will be applicable to both native and modified insulin. A major advantage of this molecule is that it provides a long acting insulin which is partially in an inactive form providing a pharmacokinetic profile that trends towards zero order biological kinetics and reducing the risk of hypoglycaemia.

According to an aspect of the invention there is provided a nucleic acid molecule comprising a nucleic acid sequence that encodes a polypeptide that has the activity of insulin wherein said polypeptide comprises insulin, or a receptor binding part thereof, linked directly or indirectly, to the insulin binding domain of the insulin receptor.

According to an aspect of the invention there is provided a fusion polypeptide comprising: the amino acid sequence of an insulin polypeptide, or an active receptor binding part thereof, linked directly or indirectly, to an insulin receptor polypeptide.

In a preferred embodiment of the invention said insulin polypeptide is native insulin; preferably human insulin.

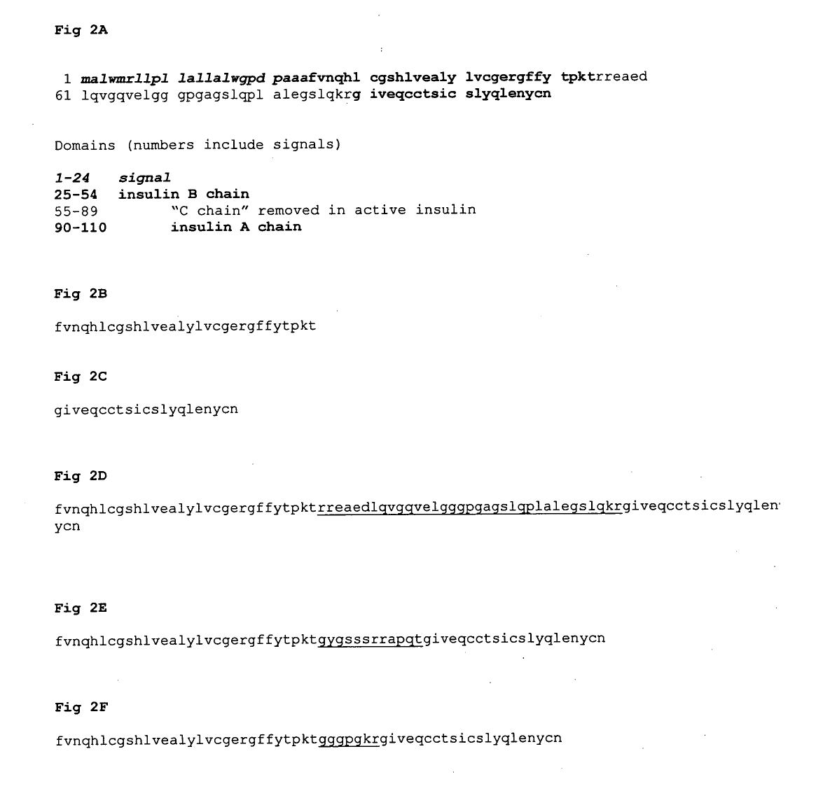

In a preferred embodiment of the invention said insulin polypeptide comprises or consists of the amino acid sequence represented in FIG. 2a, 2b, 2c, 2d, 2e, or 2f.

In an alternative preferred embodiment of the invention said insulin polypeptide is modified insulin.

“Modified insulin” represents a sequence variant of native insulin. Modified sequence variants are known in the art and include commercially available variants such as aspart, lipspro, lente, ultralente, glargine and determir.

In a preferred embodiment of the invention insulin is linked to the binding domain of the of the insulin receptor by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5, 6, 7, 8 9 or 10 copies of the peptide Gly Gly Gly Gly Ser.

Preferably, said peptide linking molecule consists of 4 copies of the peptide Gly Gly Gly Gly Ser.

Preferably, said peptide linking molecule consists of 8 copies of the peptide Gly Gly Gly Gly Ser.

In a still further alternative embodiment of the invention said polypeptide does not comprise a peptide linking molecule and is a direct fusion of insulin and the insulin binding domain of the insulin receptor.

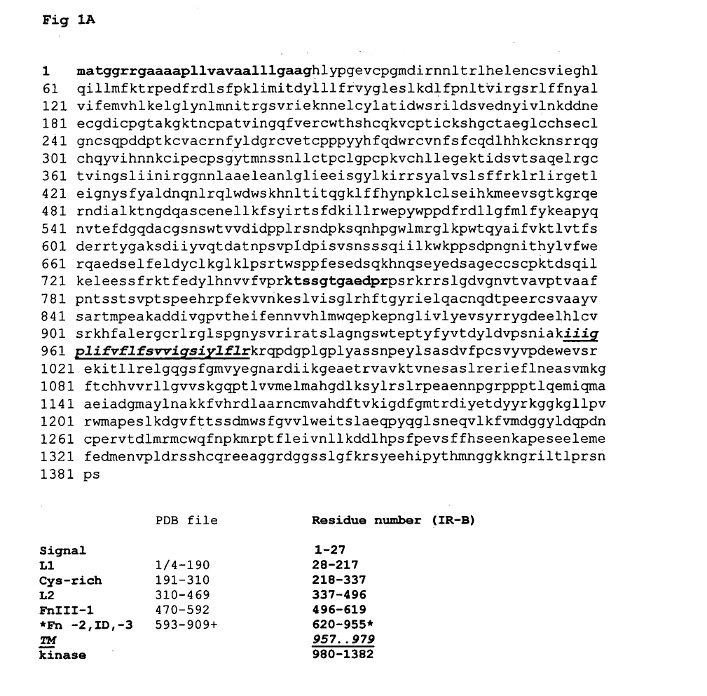

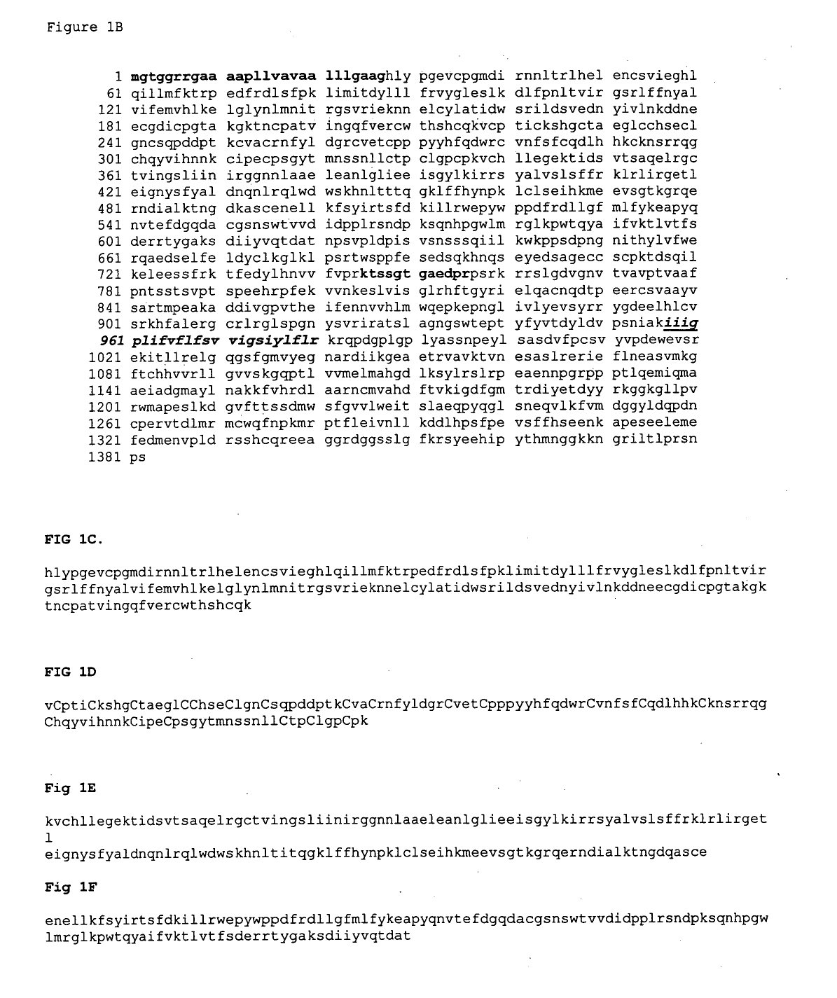

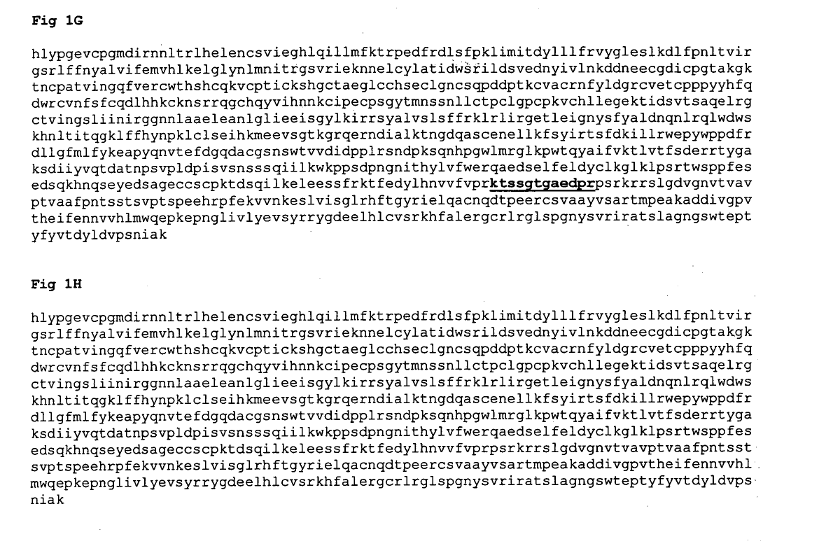

The insulin receptor and its binding domain include polymorphic sequence variants which are within the scope of the invention. For example with reference to FIG. 1i residue 448 is threonine (T), and 492 is lysine (K) but can be isoleucine (I) and glutamine (Q) respectively. Other polymorphisms in the gene encoding human insulin receptor the resulting in amino acid changes include: G 58->R; Y 171->H; G 811->S; and P 830->L.

In a preferred embodiment of the invention said insulin receptor polypeptide comprises or consists of an amino acid sequence selected from the group consisting of: FIG. 1a, 1b, 1c, 1d, 1e, 1f, 1g or 1h.

The amino acid sequences presented in FIGS. 1a-1h describe insulin receptor polypeptides and domains of insulin receptor polypeptides. The presence of a peptide signal sequence [as indicated in bold at the amino terminal end of the sequence] is optional and this disclosure relates to sequences with and without signal sequences. This applies mutatis mutandis to sequences herein disclosed that include signal sequences.

In a preferred embodiment of the invention said insulin receptor polypeptide consists of the amino acid sequence in FIG. 1g or 1h.

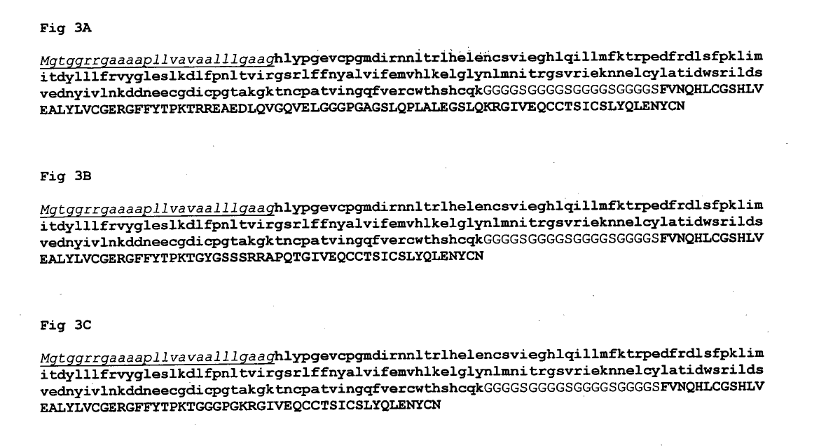

In a preferred embodiment of the invention said fusion polypeptide comprises or consists of an amino acid sequence as represented in FIG. 3a wherein said polypeptide has insulin receptor modulating activity.

In a preferred embodiment of the invention said fusion polypeptide comprises or consists of an amino acid sequence as represented in FIG. 3b wherein said polypeptide has insulin receptor modulating activity.

In a preferred embodiment of the invention said fusion polypeptide comprises or consists of an amino acid sequence as represented in FIG. 3c wherein said polypeptide has insulin receptor modulating activity.

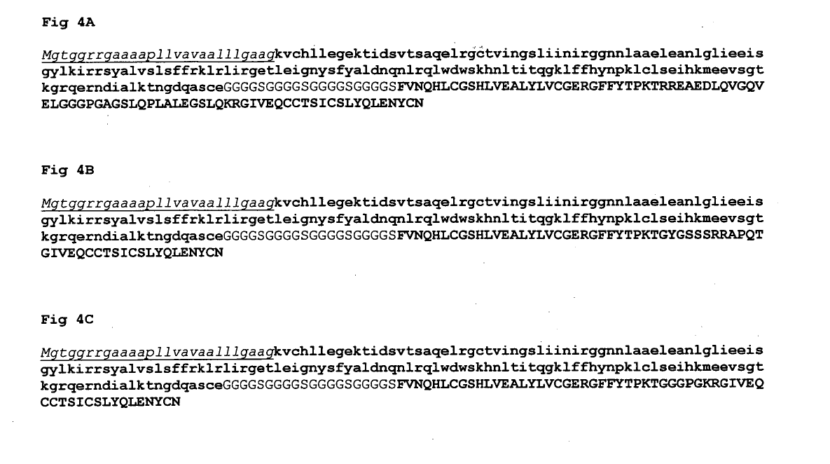

In a preferred embodiment of the invention said fusion polypeptide comprises or consists of an amino acid sequence as represented in FIG. 4a wherein said polypeptide has insulin receptor modulating activity.

In a preferred embodiment of the invention said fusion polypeptide comprises or consists of an amino acid sequence as represented in FIG. 4b wherein said polypeptide has insulin receptor modulating activity.

In a preferred embodiment of the invention said fusion polypeptide comprises or consists of an amino acid sequence as represented in FIG. 4c wherein said polypeptide has insulin receptor modulating activity.

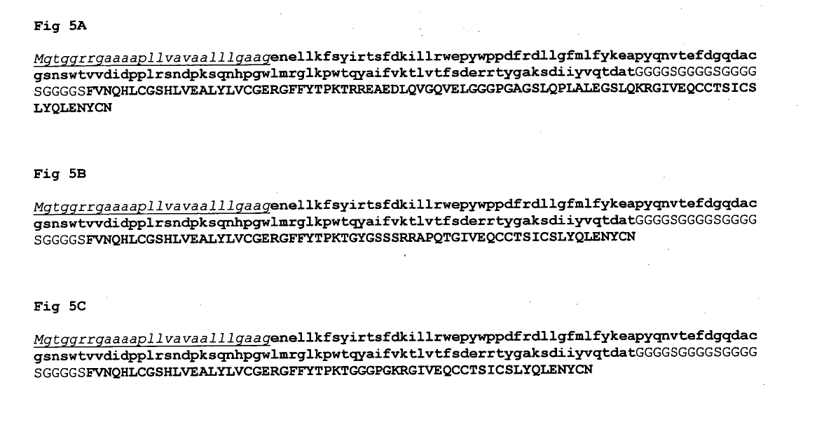

In a preferred embodiment of the invention said fusion polypeptide comprises or consists of an amino acid sequence as represented in FIG. 5a wherein said polypeptide has insulin receptor modulating activity.

In a preferred embodiment of the invention said fusion polypeptide comprises or consists of an amino acid sequence as represented in FIG. 5b wherein said polypeptide has insulin receptor modulating activity.

In a preferred embodiment of the invention said fusion polypeptide comprises or consists of an amino acid sequence as represented in FIG. 5c wherein said polypeptide has insulin receptor modulating activity.

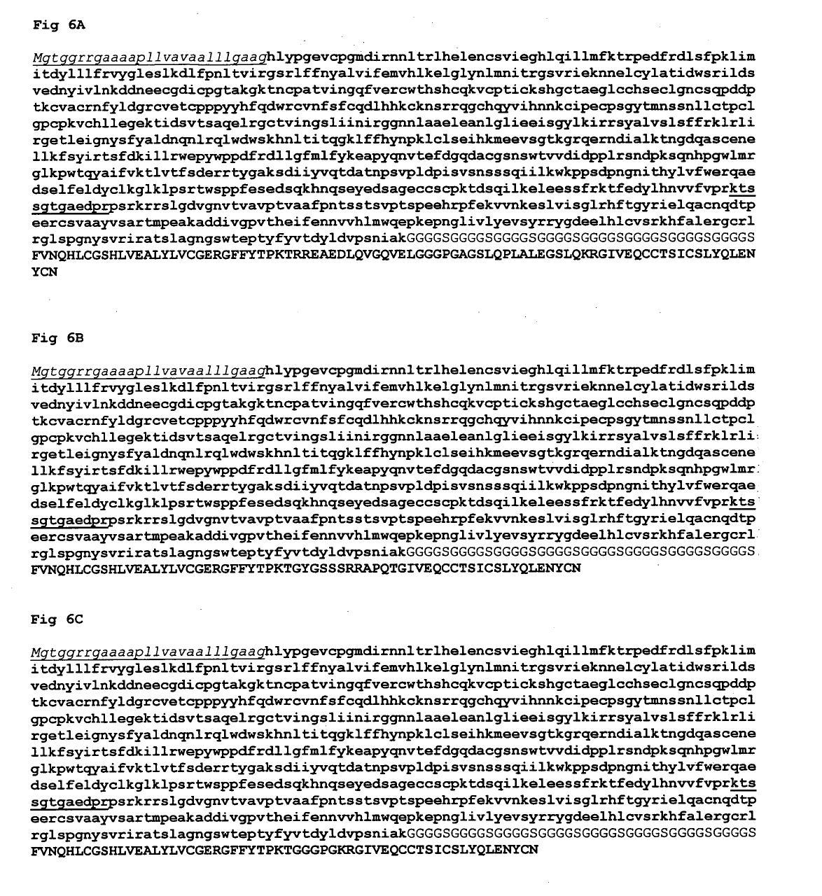

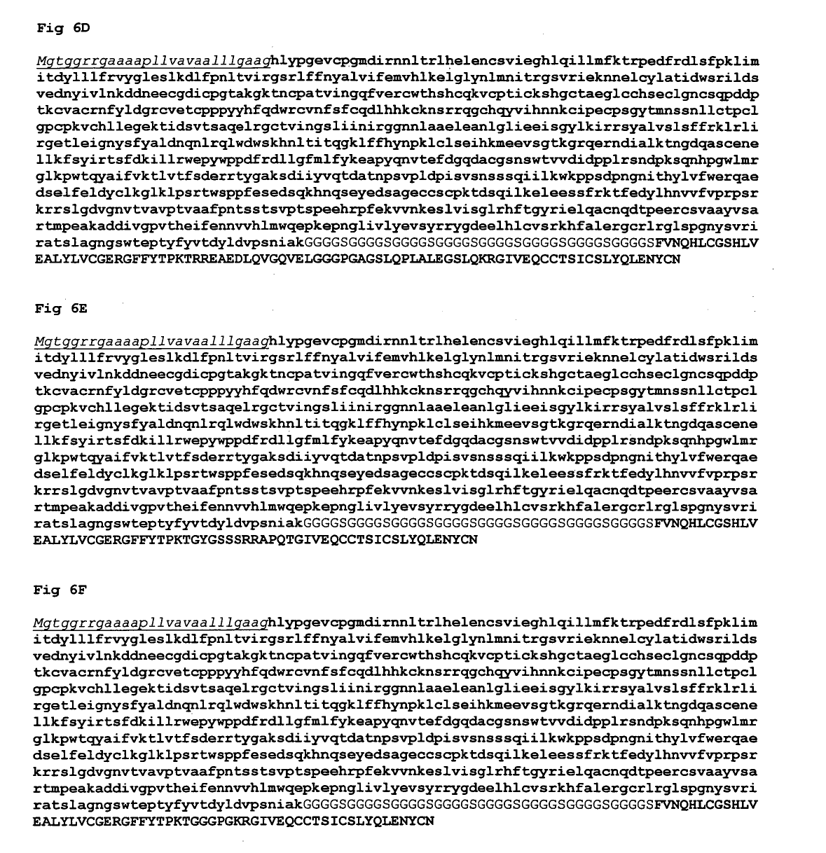

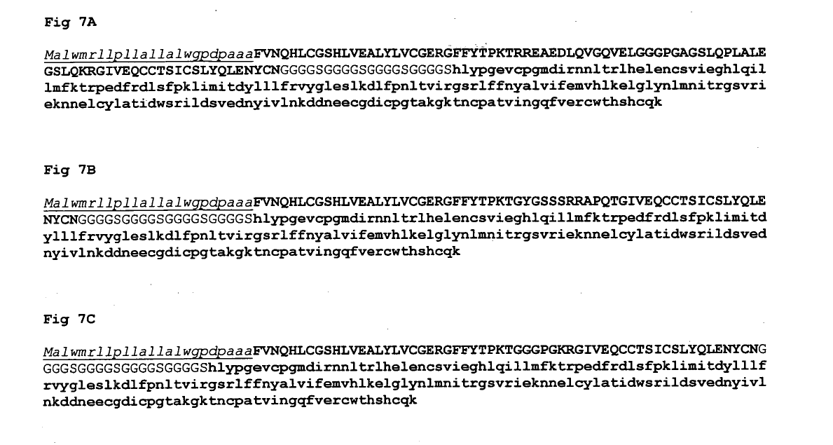

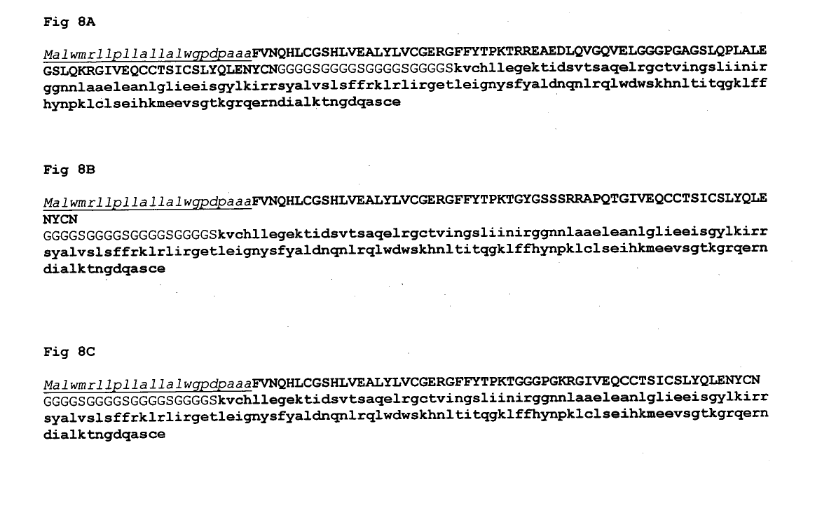

In a preferred embodiment of the invention said fusion polypeptide comprises or consists of an amino acid sequence as represented in FIG. 6a wherein said polypeptide has insulin receptor modulating activity.

In a preferred embodiment of the invention said fusion polypeptide comprises or consists of an amino acid sequence as represented in FIG. 6b wherein said polypeptide has insulin receptor modulating activity.

In a preferred embodiment of the invention said fusion polypeptide comprises or consists of an amino acid sequence as represented in FIG. 6c wherein said polypeptide has insulin receptor modulating activity.

In a preferred embodiment of the invention said fusion polypeptide comprises or consists of an amino acid sequence as represented in FIG. 6d wherein said polypeptide has insulin receptor modulating activity.

In a preferred embodiment of the invention said fusion polypeptide comprises or consists of an amino acid sequence as represented in FIG. 6e wherein said polypeptide has insulin receptor modulating activity.

In a preferred embodiment of the invention said fusion polypeptide comprises or consists of an amino acid sequence as represented in FIG. 6f wherein said polypeptide has insulin receptor modulating activity.

In a preferred embodiment of the invention said fusion polypeptide comprises or consists of an amino acid sequence as represented in FIG. 7a wherein said polypeptide has insulin receptor modulating activity.

In a preferred embodiment of the invention said fusion polypeptide comprises or consists of an amino acid sequence as represented in FIG. 7b wherein said polypeptide has insulin receptor modulating activity.

In a preferred embodiment of the invention said fusion polypeptide comprises or consists of an amino acid sequence as represented in FIG. 7c wherein said polypeptide has insulin receptor modulating activity.

In a preferred embodiment of the invention said fusion polypeptide comprises or consists of an amino acid sequence as represented in FIG. 8a wherein said polypeptide has insulin receptor modulating activity.

In a preferred embodiment of the invention said fusion polypeptide comprises or consists of an amino acid sequence as represented in FIG. 8b wherein said polypeptide has insulin receptor modulating activity.

In a preferred embodiment of the invention said fusion polypeptide comprises or consists of an amino acid sequence as represented in FIG. 8c wherein said polypeptide has insulin receptor modulating activity.

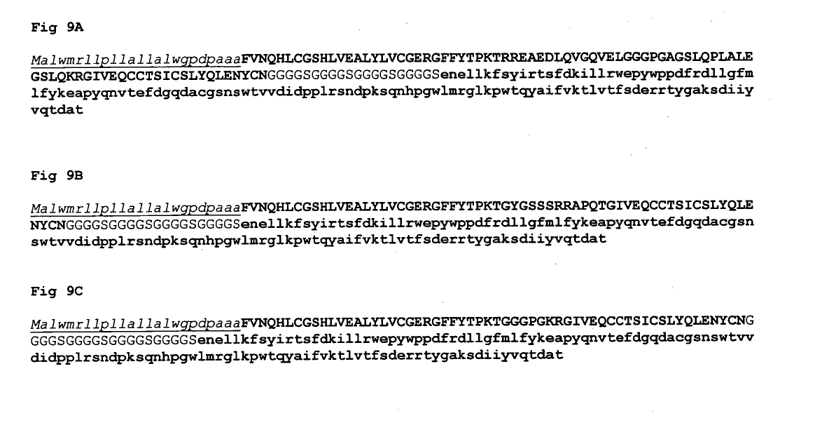

In a preferred embodiment of the invention said fusion polypeptide comprises or consists of an amino acid sequence as represented in FIG. 9a wherein said polypeptide has insulin receptor modulating activity.

In a preferred embodiment of the invention said fusion polypeptide comprises or consists of an amino acid sequence as represented in FIG. 9b wherein said polypeptide has insulin receptor modulating activity.

In a preferred embodiment of the invention said fusion polypeptide comprises or consists of an amino acid sequence as represented in FIG. 9c wherein said polypeptide has insulin receptor modulating activity.

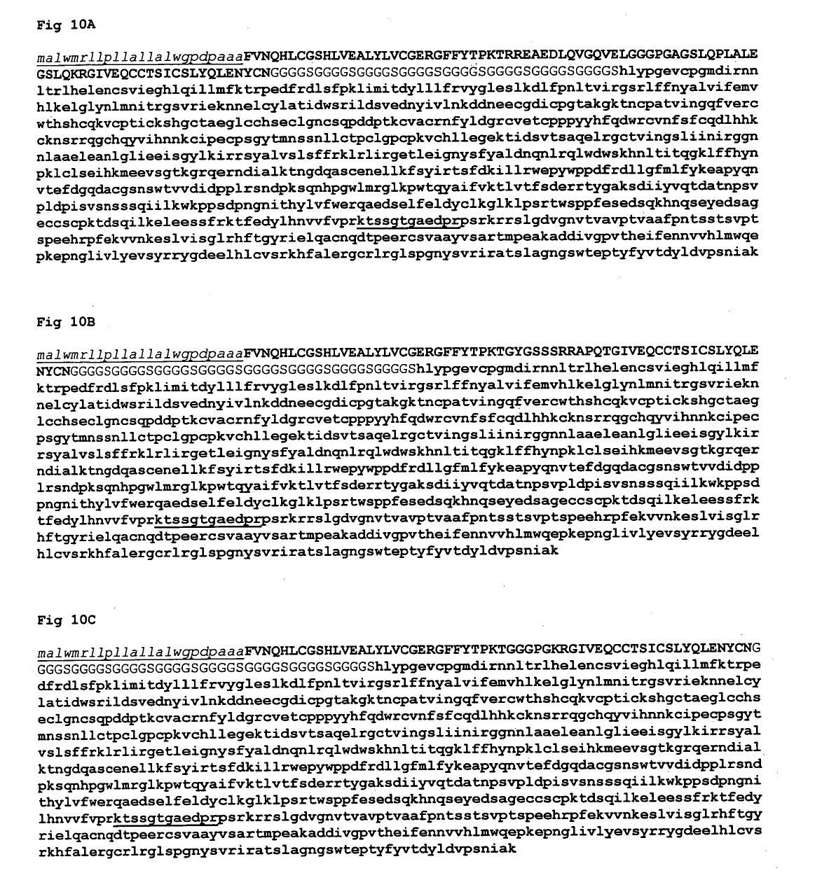

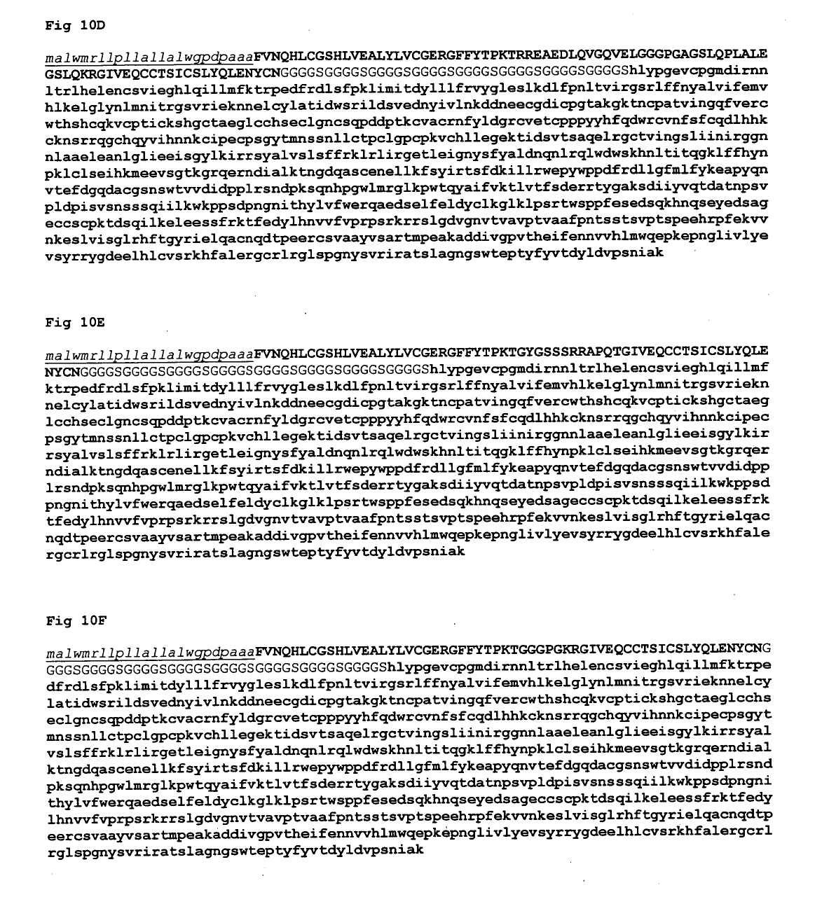

In a preferred embodiment of the invention said fusion polypeptide comprises or consists of an amino acid sequence as represented in FIG. 10a wherein said polypeptide has insulin receptor modulating activity.

In a preferred embodiment of the invention said fusion polypeptide comprises or consists of an amino acid sequence as represented in FIG. 10b wherein said polypeptide has insulin receptor modulating activity.

In a preferred embodiment of the invention said fusion polypeptide comprises or consists of an amino acid sequence as represented in FIG. 10c wherein said polypeptide has insulin receptor modulating activity.

In a preferred embodiment of the invention said fusion polypeptide comprises or consists of an amino acid sequence as represented in FIG. 10d wherein said polypeptide has insulin receptor modulating activity.

In a preferred embodiment of the invention said fusion polypeptide comprises or consists of an amino acid sequence as represented in FIG. 10e wherein said polypeptide has insulin receptor modulating activity.

In a preferred embodiment of the invention said fusion polypeptide comprises or consists of an amino acid sequence as represented in FIG. 10f wherein said polypeptide has insulin receptor modulating activity.

In a preferred embodiment of the invention said polypeptide is an agonist.

In an alternative preferred embodiment of the invention said polypeptide is an antagonist.

According to a further aspect of the invention there is provided a nucleic acid molecule that encodes a polypeptide according to the invention.

According to an aspect of the invention there is provided a homodimer consisting of two polypeptides according to the invention.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

In a preferred embodiment of the invention said further therapeutic agent is a modified insulin variant.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceutical compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from hyperglycaemia comprising administering an effective amount of at least one polypeptide according to the invention.

According to a further aspect of the invention there is provided a method to treat a human subject suffering from hypoglycaemia comprising administering an effective amount of at least one polypeptide according to the invention.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

In a preferred method of the invention said hyperglycaemic condition is diabetes mellitus.

In a preferred method of the invention diabetes mellitus is type 1.

In a preferred method of the invention diabetes mellitus is type 2.

In a preferred method of the invention said hyperglycaemia is the result of insulin resistance.

In a preferred method of the invention said hyperglycaemia is the result of Metabolic Syndrome.

According to an aspect of the invention there is provided the use of a polypeptide according to the invention for the manufacture of a medicament for the treatment of diabetes mellitus.

In a preferred embodiment of the invention diabetes mellitus is type 1.

In a preferred embodiment of the invention diabetes mellitus is type 2.

In a preferred method of the invention said hyperglycaemia is the result of insulin resistance.

In a preferred embodiment of the invention said hyperglycaemia is the result of Metabolic Syndrome.

In a further preferred embodiment of the invention said polypeptide is administered at two day intervals; preferably said polypeptide is administered at weekly, 2 weekly or monthly intervals.

According to a further aspect of the invention there is provided a monoclonal antibody that binds the polypeptide or dimer according to the invention.

Preferably said monoclonal antibody is an antibody that binds the polypeptide or dimer but does not specifically bind insulin or insulin receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1A illustrates human insulin receptor isoform IR-A; FIG. 1B illustrates human insulin receptor isoform IR-B FIG. 1C is the L1 domain of human insulin receptor; FIG. 1D is the cystiene rich domain of human insulin receptor; FIG. 1E is the L2 sub-domain of human insulin receptor; FIG. 1F is the FnIII-1 domain of human insulin receptor; FIG. 1G is the extracellular domain of human insulin receptor isoform B [amino acids 28-955]; FIG. 1H is the extracellular domain of human insulin receptor isoform A [amino acids 28-943] FIG. 1i is the human insulin receptor illustrating polymorphic variant sequences;

FIG. 2A is the amino acid sequence of human insulin precursor including a summary of the sub-domains; FIG. 2B is the amino acid sequence of human insulin chain B; FIG. 2C is the amino acid sequence of human insulin chain A; FIG. 2D is the amino acid sequence of human proinsulin; FIG. 2E is the amino acid sequence of peptide linked B and A chains of human insulin 1; FIG. 2F is the amino acid sequence of peptide linked A and B chains of human insulin 2;

FIG. 3A is a chimeric fusion protein comprising of receptor L1 domain and proinsulin; FIG. 3B is a chimeric fusion protein comprising of receptor L1 domain and single chain insulin 1; FIG. 3C is a chimeric fusion protein comprising of receptor L1 domain and single chain insulin 2;

FIG. 4A is a chimeric fusion protein comprising of receptor L2 domain and proinsulin; FIG. 4B is a chimeric fusion protein comprising of receptor L2 domain and single chain insulin 1; FIG. 4C is a chimeric fusion protein comprising of receptor domain L2 and single chain insulin 2;

FIG. 5A is a chimeric fusion protein comprising of receptor FnIII-1 domain and proinsulin; FIG. 5B is a chimeric fusion protein comprising FnIII-1 domain and single chain insulin 1; FIG. 5C is a chimeric fusion protein comprising FnIII-1 domain and single chain insulin 2;

FIG. 6A is a chimeric fusion protein comprising of the extracellular domain of insulin receptor isoform B and proinsulin; FIG. 6B is a chimeric fusion protein comprising the extracellular domain of insulin receptor isoform B and single chain insulin 1; FIG. 6C is a chimeric fusion protein comprising the extracellular domain of insulin receptor isoform B and single chain insulin 2; FIG. 6D is a chimeric fusion protein comprising the extracellular domain of insulin receptor isoform A and proinsulin; FIG. 6E is a chimeric fusion protein comprising the extracellular domain of insulin receptor isoform A and single chain insulin 1: FIG. 6F is a chimeric fusion protein comprising the extracellular domain of insulin receptor isoform A and single chain insulin 2;

FIG. 7A is a chimeric fusion protein comprising proinsulin and insulin receptor domain L1; FIG. 7B is a chimeric fusion protein comprising single chain insulin 1 and insulin receptor domain L1; FIG. 7C is a chimeric fusion protein comprising single chain insulin 1 and insulin receptor domain L1;

FIG. 8A is a chimeric fusion protein comprising proinsulin and insulin receptor domain L2; FIG. 8B is a chimeric fusion protein comprising single chain insulin 1 and insulin receptor domain L2; FIG. 8C is a chimeric fusion protein comprising single chain insulin 1 and insulin receptor domain L2;

FIG. 9A is a chimeric fusion protein comprising proinsulin and insulin receptor FnIII-1 domain; FIG. 9B is a chimeric fusion protein comprising single chain insulin 1 and insulin receptor FnIII-1 domain; FIG. 9C is a chimeric fusion protein comprising single chain insulin 2 and insulin receptor FnIII-1 domain;

FIG. 10A is a chimeric fusion protein comprising proinsulin and the extracellular domain of insulin isoform B; FIG. 10B is a chimeric fusion protein comprising single chain insulin 1 and the extracellular domain of insulin isoform B; FIG. 10C is a chimeric fusion protein comprising single chain insulin 2 and the extracellular domain of insulin isoform B; FIG. 10D is a chimeric fusion protein comprising proinsulin and the extracellular domain of insulin isoform A; FIG. 10E is a chimeric fusion protein comprising single chain insulin 1 and the extracellular domain of insulin isoform A; FIG. 10F is a chimeric fusion protein comprising single chain insulin 2 and the extracellular domain of insulin isoform A;

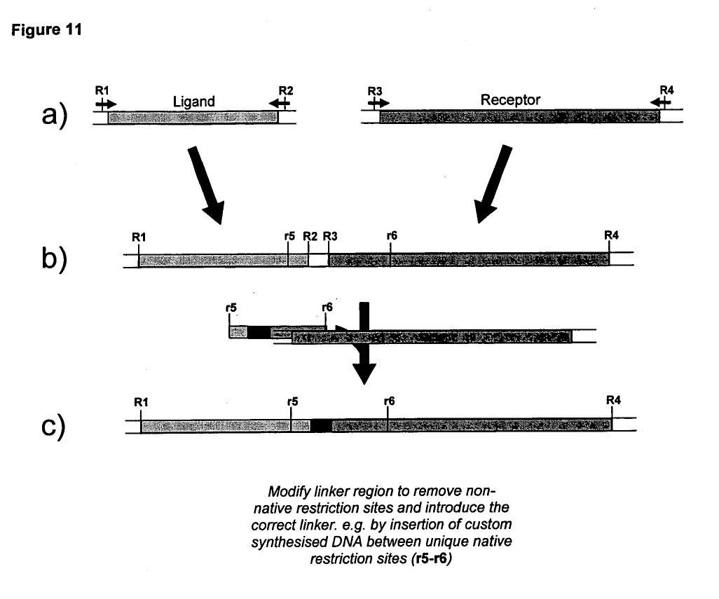

FIG. 11 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites);

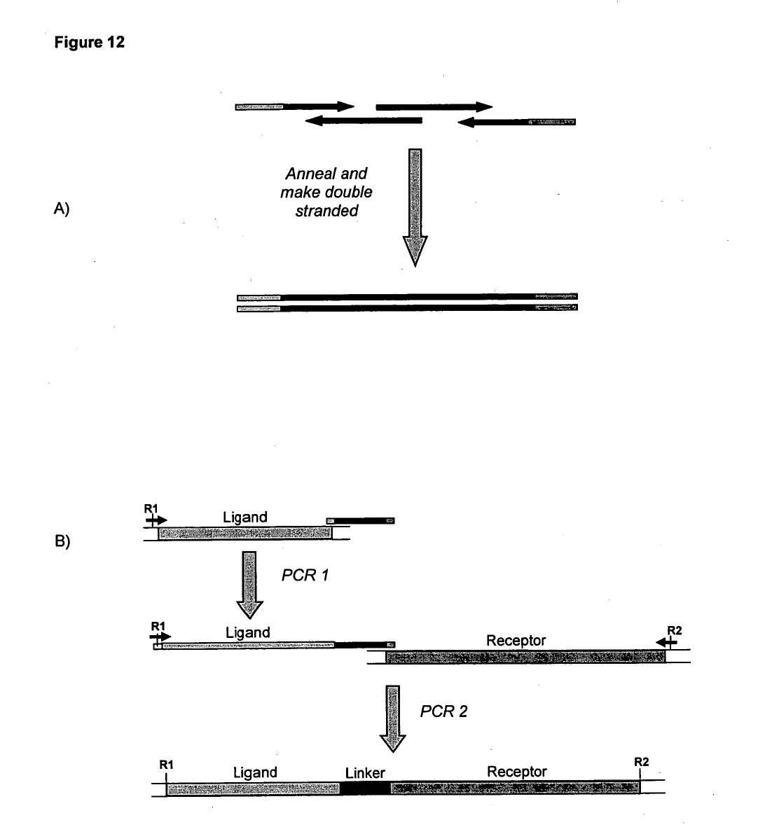

FIG. 12 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector; and

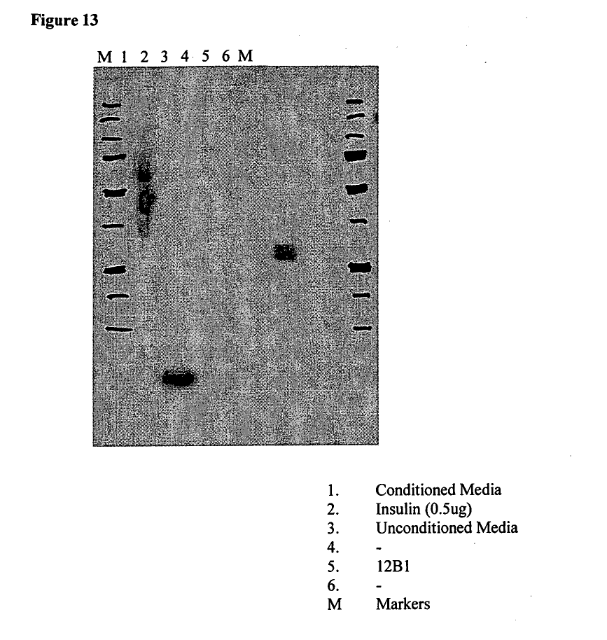

FIG. 13 expression and immune blot of insulin fusion protein 12B1

### MATERIALS AND METHODS

### Testing for Insulin Fusion Protein Activity

Methods for testing the biological activity of insulin fusion proteins herein described are well known in the art. For example methods and assays described in US2008/057004, US2006/286182, US2005/171008 or U.S. Pat. No. 6,200,569 each of which is incorporated by reference.

### Immunological Testing

Immunoassays that measure the binding of insulin to polyclonal and monoclonal antibodies are known in the art. Commercially available insulin antibodies are available to detect insulin in samples and also for use in competitive inhibition studies. For example monoclonal antibodies can be purchased at http://www.ab-direct.com/index AbD Serotec.

### Recombinant Production of Fusion Proteins

The components of the fusion proteins were generated by PCR using primers designed to anneal to the ligand or receptor and to introduce suitable restriction sites for cloning into the target vector (FIG. 11a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 11b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 11c).

Alternatively, the linker with flanking sequence, designed to anneal to the ligand or receptor domains of choice, was initially synthesised by creating an oligonucleotide duplex and this processed to generate double-stranded DNA (FIG. 12a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 12b).

### Expression and Purification of Fusion Proteins

Expression was carried out in a suitable system (e.g. mammalian CHO cells, E. coli,) and this was dependant on the vector into which the insulin-fusion gene was generated. Expression was then analysed using a variety of methods which could include one or more of SDS-PAGE, Native PAGE, western blotting, ELISA well known in the art.

Once a suitable level of expression was achieved the insulin fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

### Characterisation of Insulin-Fusions

Denaturing PAGE, native PAGE gels and western blotting were used to analyse the fusion polypeptides and western blotting performed with antibodies non-conformationally sensitive to the insulin fusion. Native solution state molecular weight information can be obtained from techniques such as size exclusion chromatography using a Superose G200 analytical column and analytical ultracentrifugation.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

Insulin LR-Fusion Expression: Western Blot of 12B1 from Stable Expressions in CHO Flpln Cells.

1 ml of sample concentrated and then run on and SDS-PAGE gel (Lane 2). Conditioned and unconditioned media were also concentrated and run on the gel. Markers are at 250, 150, 100, 75, 50, 37, 25, 20 and 15 kDa. Immunoblot carried out with mouse anti-insulin antibody (Abcam.; Cat#: ab9569; dilution=1:100) and anti-mouse-HRP antibody (Abcam; dilution=1:2500).

1. A nucleic acid molecule comprising a nucleic acid sequence that encodes a polypeptide that has the activity of human insulin wherein said polypeptide comprises human insulin to a human insulin receptor polypeptide.

2. A fusion polypeptide comprising: the amino acid sequence of a human insulin polypeptide linked to a human insulin receptor polypeptide.

3. A fusion polypeptide according to claim 2, wherein said human insulin polypeptide is native human insulin.

4. (canceled)

5. A fusion polypeptide according to claim 1, wherein said human insulin polypeptide comprises the amino acid sequence represented in FIG. 2a, 2b, 2c, 2d, 2e, or 2f.

6. A fusion polypeptide according to claim 2, wherein said insulin polypeptide is modified human insulin.

7. A fusion polypeptide according to claim 1 wherein the human insulin is linked to a binding domain of the insulin receptor by a peptide linker.

8. A fusion polypeptide according to claim 7, wherein said peptide linker is a flexible peptide linker.

9. A fusion polypeptide according to claim 8, wherein said peptide linker comprises at least one copy of the peptide Gly Gly Gly Gly Ser.

10. A fusion polypeptide according to claim 9, wherein said peptide linker comprises 2, 3, 4, 5, 6, 7, 8, 9 or 10 copies of the peptide Gly Gly Gly Gly Ser.

11. A fusion polypeptide according to claim 9, wherein said peptide linker consists of 4 copies of the peptide Gly Gly Gly Gly Ser.

12. A fusion polypeptide according to claim 9, wherein said peptide linker consists of 8 copies of the peptide Gly Gly Gly Gly Ser.

13. A fusion polypeptide according to claim 2, wherein said polypeptide does not comprise a peptide linker and is a direct fusion of human insulin and the human insulin receptor polypeptide.

14. A fusion polypeptide according to claim 1, wherein said human insulin receptor polypeptide comprises the amino acid sequence as represented in FIG. 1a, 1b, 1c, 1d, 1e, 1f, 1g or 1h.

15. A fusion polypeptide according to claim 14, wherein said human insulin receptor polypeptide consists of the amino acid sequence as represented in FIG. 1g or 1h.

16. A fusion polypeptide according to claim 2, wherein said fusion polypeptide comprises the amino acid sequence as represented in FIG. 3a, 3b or 3c wherein said fusion polypeptide has insulin receptor modulating activity.

17-18. (canceled)

19. A fusion polypeptide according to claim 2, wherein said fusion polypeptide comprises the amino acid sequence as represented in FIG. 4a, 4b or 4c, wherein said polypeptide has insulin receptor modulating activity.

20-21. (canceled)

22. A fusion polypeptide according to claim 2, wherein said fusion polypeptide comprises the amino acid sequence as represented in FIG. 5a, 5b or 5c, wherein said polypeptide has insulin receptor modulating activity.

23-24. (canceled)

25. A fusion polypeptide according to claim 2, wherein said fusion polypeptide comprises the amino acid sequence as represented in FIG. 6a, 6b, 6c, 6d or 6f, wherein said polypeptide has insulin receptor modulating activity.

26-30. (canceled)

31. A fusion polypeptide according to claim 2, wherein said fusion polypeptide comprises the amino acid sequence as represented in FIG. 7a, 7b or 7c, wherein said polypeptide has insulin receptor modulating activity.

32-33. (canceled)

34. A fusion polypeptide according to claim 2, wherein said fusion polypeptide comprises the amino acid sequence as represented in FIG. 8a, 8b or 8c, wherein said polypeptide has insulin receptor modulating activity.

35-36. (canceled)

37. A fusion polypeptide according to claim 2, wherein said fusion polypeptide comprises the amino acid sequence as represented in FIG. 9a, 9b or 9c, wherein said polypeptide has insulin receptor modulating activity.

38-39. (canceled)

40. A fusion polypeptide according to claim 2, wherein said fusion polypeptide comprises the amino acid sequence as represented in FIG. 10a, 10b, 10c, 10d, 10e or 10f wherein said polypeptide has insulin receptor modulating activity.

41-48. (canceled)

49. A vector comprising the nucleic acid molecule according to claim 1.

50. A cell transfected or transformed with the vector according to claim 49.

51. A homodimer consisting of two fusion polypeptides according to claim 2.

52. A pharmaceutical composition comprising a fusion polypeptide according to claim 2 and an excipient or carrier.

53. (canceled)

54. A method to treat a human subject suffering from hyperglycemia, comprising administering an effective amount of at least one polypeptide according to claim 2, thereby treating the hyperglycemia in the subject.

55-66. (canceled)

67. A method according to claim 54, wherein said effective amount is administered at two day, weekly, 2-weekly or monthly intervals.
